SEQUENCE LISTING (6 sequences)

SEQ ID NO: 1 - DNA, 732 bp, artificial sequence
  fusion sequence of Vh of anti-collgenase IV and LDP
atgcaggtgaagctgcagcagtctggaactgaagtggtaaagcctggggcttcagtgaagttgtcctgcaaggcttctggctacatcttcacaagttatgatatagactgggtgaggcagacgcctgaacagggacttgagtggattggatggatttttcctggagaggggagtactgaatacaatgagaagttcaagggcagggccacactgagtgtagacaagtcctccagcacagcctatatggagctcactaggctgacatctgaggactctgctgtctatttctgtgctagaggggactactataggcgctactttgacttgtggggccaagggaccacggtcaccgtctcctcaggcggtggcggatccgcgcccgccttctccgtcagtcccgcctcgggtctgagtgacggacagagcgtgtcggtgtcggtcagcggtgccgccgccggcgagacctactacatcgcccagtgcgctccggtcggtggccaggacgcgtgcaacccggcgaccgcgacgtccttcaccacggacgcgtccggagcggcgtcgttcagcttcgtcgtgcgcaagtcgtacacgggctccacgcccgaaggcacgccggtcggcagcgtcgactgcgccacggccgcctgtaacctcggcgccggcaactccgggctcgacctcggccacgtggctctgaccttcggcctcgagcaccaccaccaccaccactga

SEQ ID NO: 2 - protein, 243 aa, artificial sequence
  fusion protein of Vh and LDP
MQVKLQQSGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSSGGGGSAPAFSVSPASGLSDGQSVSVSVSGAAAGETYYIAQCAPVGGQDACNPATATSFTTDASGAASFSFVVRKSYTGSTPEGTPVGSVDCATAACNLGAGNSGLDLGHVALTFGLEHHHHHH

SEQ ID NO: 3 - DNA, 31 bp, artificial sequence
  primer
gatacatatgcaggtgaagctgcagcagtct

SEQ ID NO: 4 - DNA, 40 bp, artificial sequence
  primer
cataggatccgccaccgcctgaggagacggtgaccgtggt

SEQ ID NO: 5 - DNA, 31 bp, artificial sequence
  primer
gataggatccgcgcccgccttctccgtcagt

SEQ ID NO: 6 - DNA, 27 bp, artificial sequence
  primer
ctcgaggccgaaggtcagagccacgtg

(19) United States
(12) Patent Application Publication
(10) Pub. No.: US 2008/0044412 A1
(43) Pub. Date: Feb. 21, 2008

(54) Single-Domain Antibody Strengthening Fusion Protein Vh-Ldp-Ae

(75) Inventors: Qingfang Miao, Beijing (CN); Shuzhen Chen, Beijing (CN); Yongsu Zhen, Beijing (CN); Boyang Shang, Beijing (CN); Xiujun Liu, Beijing (CN); Xiaoyun Liu, Beijing (CN)

Correspondence Address: LADAS & PARRY, 26 WEST 61ST STREET, NEW YORK, NY, 10023, US

(73) Assignee: INSTITUTE OF MEDICINAL BIOTECHNOLOGY CHINESE ACADEMY OF MEDICAL SCIENCES, BEIJING, SWITZERLAND (CH)

(21) Appl. No.: 11/587,199

(22) PCT Filed: Apr. 19, 2005
(86) PCT No.: PCT/CN05/00534

§ 371 Date: Sep. 24, 2007

(30) Foreign Application Priority Data
Apr. 23, 2004 (CN) 200410034052.7

(51) Int. Cl. A61K 39/395; A61P 35/00; C07K 19/00; C12N 15/00

(52) U.S. Cl. 424/134.1; 435/69.7; 530/387.3

(57) ABSTRACT

The present invention relates to a novel antibody-based targeting drug—a single domain antibody energized fusion protein VH-LDP-AE with potent cancer cell killing activity, anti-angiogenic activity, and anti-cancer therapeutic efficacy, to a method for producing the same, and use thereof in manufacture of an anti-tumor medicament.

### FIELD OF THE INVENTION

The present invention relates to a novel antibody-based, targeted drug with potent cancer cell killing activity, anti-angiogenic effects and antitumor efficacy—a single domain antibody energized fusion protein VH-LDP-AE, to a method for producing the same, and use thereof in manufacture of an anti-tumor medicament.

### BACKGROUND OF THE INVENTION

Type IV collagenases can degrade the extracellular matrix components such as type IV collagen, destroy the integrality of basement membrane and extracellular matrix and play an important role in tumor growth, invasion and metastasis. There is a high expression in various kinds of tumor tissue and cells such as human prostate cancer, colorectal cancer, breast cancer, melanoma, pancreatic cancer, etc., and the inhibition of type IV collagenase activity can suppress the growth and metastasis of tumor. The present invention relates to a single domain antibody which derived from the monoclonal antibody 3G11 directed against type IV collagenase, which is immunoreactivitily positive to various kinds of tumor cells and has the capability of binding specifically to various kinds of human tumor tissues.

Single domain antibodies (dAbs) are the smallest functional binding units of an antibody, which corresponds to the variable regions of the heavy (VH) or the light (VL) chains of human antibodies. As the new generation of therapeutic antibodies after intact antibodies, Fab fragments, and single chain antibodies, single domain antibodies have a molecular weight of 11˜13 kDa, or approximately one-twelfth the size of a full antibody (150 kDa). Compared with intact antibodies, single domain antibodies have much smaller molecular size and this property determined that they not only can better penetrate into the intercellular space of solid tumor, but also own more uniform bio-distribution and lower immunogenicity in tumor. However, due to the lack of Fc portion and hence loss of the capability of trigger effector functions, dAbs need to be used in combination with the “warhead” drug in cancer therapy. DAbs are promising as an excellent vehicle candidate for targeted therapy of cancer.

As the highly potent “warhead” drug, lidamycin (LDM, also named C-1027 or C1027) is an enediyne-containing antibiotic produced by Streptomyces globisporus (accession number: CGMCC No. 0135) which was isolated from a soil sample collected in Qianjiang County, Hubei Province, China. LDM is one of the macromolecular peptide antibiotics which displays the most potent cytotoxicity against tumor cells reported hitherto. In vivo experiments had demonstrated that LDM remarkably inhibited the growth of colon carcinoma 26 in mice, and also showed potent inhibitory effect on the growth of human cancer xenografts such as hepatoma Bel-7402 and cecum carcinoma Hce-8693 in nude mice (Chinese Journal of Antibiotics 1994, 19 (2):164-168). The LDM molecule consists of two moieties, one is enediyne chromophore (AE, active enediyne) which is labile and responsible for cytotoxicity; another is apoprotein (LDP) containing 110 amino acid residues, which plays a pivotal role in keeping the stability of chromophore. There is a non-covalent binding between chromophore and apoprotein. Although the binding is specific and fast, the chromomhore and apoprotein can be separated and molecular reconstituted. Due to the unique molecular structure, LDM is a perfect “warhead” candidate for constructing novel antibody-based, targeting drugs (Acta Academiae Medicinae Sinicae, 2001, 23 (6): 563-567).

The targeted cancer therapy has better efficacy because it can right deliver a drug to cancer tissue and decrease the toxicity to normal cells. Monoclonal antibodies have been used as carriers for drug in targeted cancer therapy. However, clinic studies demonstrated that the large mAb molecules were unable to penetrate tumor effectively to reach the region deep within the solid tumor; as a result, the solid tumor is relatively resistant to targeted immunotherapy of cancer. In addition, the high normal-tissue/tumor distribution ratio of large antibody molecules and high immunogenecity of mouse antibody both limited their clinical use. If minimize the antibody size while preserve its antigen affinity, and at the same time, load it with “warhead” drug which has potent cytotoxic activity against tumor cells, the “down-sized” antibody molecule carrying the “warhead” drug will be able to reach efficiently the tumor target and the region deep within the solid tumor. This strategy may not only get over the resistance problem of solid tumor against antibody-based therapy and reduce the immunogencity, but also decrease the effective concentration of the “warhead” drug and make it work at much lower concentrations. So the therapeutic effects will be improved. According to the strategy, using the advantage of single domain antibody and the characteristic of LDM which can be separated and reconstituted, the present inventors manufactured a novel, down-sized, and highly effective dAb-based energized fusion protein VH-LDP-AE by gene recombination and molecular reconstitution as a novel antibody-targeted drug which demonstrates potent antitumor activity.

### DETAILED DESCRIPTION OF THE INVENTION

The conjugates of large monoclonal antibody and drug are hard to reach the tumor cells deep within the solid tumor through the endothelium of capillary vessels and extracellular space in solid tumor in tumor therapy. It is valuable to develop a down-sized and highly-effective antibody drug for improving the therapeutic efficacy (Yong-su Zhen. Advances in Research on Monoclonal Antibody Agents for Cancer Therapy. Acta Academiae Medicinae Sinicae, 2000, 22 (1): 9-13). Using VH domain of mAb 3G11 which directed against type IV collagenase as a carrier and the highly-potent antitumor antibiotic LDM as the “warhead”, the present inventors, by DNA recombination and molecular reconstitution, manufactured the novel antibody-targeted, dAb-based, and energized fusion protein VH-LDP-AE characterized as down-sized molecule and high efficacy. VH-LDP-AE can bind specifically to tumor cells, inhibit angiogenesis, and also demonstrate good antitumor efficacy in animal experiments.

In one respect, the present invention relates to the said domain-antibody-based and energized fusion protein VH-LDP-AE, which composed of fusion protein VH-LDP and the active enediyne (AE) chromophore of lidamycin, wherein the said VH-LDP protein consists of VH domain of mAb 3G11 directly against type IV collagenase, a flexible protein spacer (GGGGS), an apoprotein LDP of LDM, and six-histidine tag (His₆-Tag) in the carboxyl terminal.

1. Single-Domain Antibody Fusion Protein VH-LDP

Specifically, the gene encoding the fusion protein VH-LDP is 732 bp (SEQ ID NO: 1) and it encodes 243 amino acids (SEQ ID NO: 2). The molecular weight of VH-LDP is 25.4 kDa.

In the present invention, the VH moiety in fusion protein VH-LDP derived from the heavy chain variable region of monoclonal antibody 3G11 (hybridoma strain of 3G11 has been deposited in China General Microbiological Culture Collection Center with accession number CGMCC No. 0831). Previous experiments demonstrated that the immunoreactivity of mAb 3G11 was positive to various human tumor tissues and showed targeted distribution in human lung cancer xenograft transplanted into nude mice (Yao Dai, Bing Jia, Yong-su Zhen, et al. Immunoscintigraphy of anti-type IV collagenase monoclonal antibody in nude mice bearing human lung cancer xenograft, Chinese Journal of Cancer, 2003, 22(12): 1243-1248). The present inventors found that single domain antibody fusion protein VH-LDP owned part of antigen-binding and -inhibiting activity of the intact antibody. It can bind specifically to cancer cells and inhibit the activity of type IV collagenase.

2. The Active Enediyne Chromophore AE

The molecular weight of LDM is 11349.1120 Dalton, which is consisted of a LDP with 10505.7830 Dalton and chromophore AE with 843.3295 Dalton.

Chemical Name of Chromophore AE:

(2R,7S,9R,10R)-7-Amino-7,8-(2*-chloro-6*-hydroxy-1*,4*-phenylene)-10-(4′-deoxy-4′-dimethylamino-5′,5′-dimethyl-ribopyranosido)-4,8-dioxa-5-oxo-1,11,13-trien-15,18-diyn-tricyclo
[7,7,3,0^{10,14}]-2-nondecanyl-2″,3″-dihydro-7″-methoxy-2″-methylene-3″-oxo-1″,4″-benzo xazine-5″-carboxylate

Molecular formula of lidamycin: C₄₃H₄₂O₁₃N₃Cl

The chemical structure of active and aromatized lidamycin chromophore:

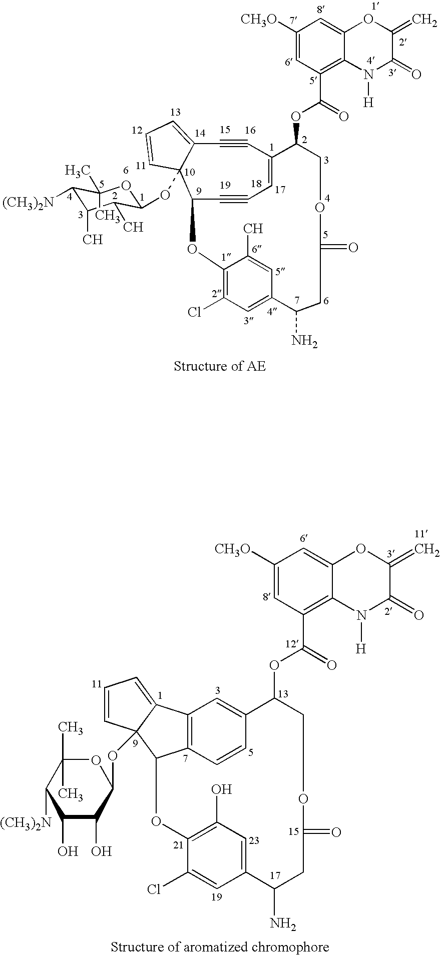

Two parts of lidamycin, LDP and chromophore, connecting with each other specifically and firmly through non-covalent binding, can be dissociated and reconstituted to rebuild an energized molecule. The unique properties of molecular constitution, low molecular weight of AE, and potent bioactivity make lidamycin a promising “warhead” agent in constructing new monoclonal antibody targeted drugs (Acta Acad Med Sin, 2001, 23<6>: 563-567).

In one embodiment of the present invention, the molar ratio of said fusion protein VH-LDP and active chromophore AE of LDM is 1:1 in the said dAb-based and energized fusion molecule VH-LDP-AE.

Another aspect of the present invention relates to the preparation of energized fusion protein VH-LDP-AE. In details, the fusion protein VH-LDP was produced at first by DNA recombination in E. coli expression system. The inventors found that the resultant fusion protein VH-LDP had the antigen-binding and antigen-inhibiting activity and can bind selectively to tumor tissue. And then, the domain-antibody-based and energized fusion protein VH-LDP-AE was obtained through assembling active domain-antibody-based fusion protein VH-LDP with active chromophore AE of lidamycin that is obtained by the method of cold methanol extraction. The molecular ratio of AE and VH-LDP is 5:1 and the volume ratio of them is 1:50. The inventors surprisingly found that VH-LDP-AE can potently kill the tumor cells and inhibit angiogenesis. At the same time, compared with the existing scFv-based energized fusion protein, VH-LDP-AE would have better penetration into solid tumors, lower immunogenicity, stronger antitumor effects, or less risk of inducing side-effects in clinical application due to its much smaller size.

On another respect, the present invention relates to use of the said energized fusion protein VH-LDP-AE in manufacture of a medicament for anti-angiogenesis and antitumor treatment.

On another respect, the present invention relates to a pharmaceutical composition comprising therapeutically effective amount of energized fusion protein VH-LDP-AE, and optionally, said pharmaceutical composition further comprises pharmaceutical acceptable carrier and excipient compatible to the administration route and dosage thereof.

On another respect, the present invention relates to a method for treating malignant cancers, including the administration of therapeutically effective amount of said energized fusion protein or the pharmaceutical composition of the present invention to the patient with tumor.

Some earlier studies showed that single VH domain can retain part of antigen affinity, but isolated VH domain tended to congregate into sedimentation. The research in the present lab demonstrated that LDM itself is water soluble, and apoprotein of lidamycin alone could be expressed solubly in E. Coli. The inventors attempted to make fusion gene in the form of VH-LDP by fusing VH domain against type IV collagenase to apoprotein LDP of LDM, and then expressing the fusion protein in E. coli. As a result, the present inventors surprisingly found that the fusion protein after refolding was soluble in PBS solution, and most of them (75%) existed in monomer state. The result showed that the constructing method in the present invention overcame effectively the adverse influence of VH congregation in solution.

As mentioned above, single-domain antibodies are the minimal functional binding units of antibody and are regarded as a new generation of therapeutic antibodies. In the present invention, using type IV collagenase as the target, single domain antibody derived from mAb 3G11 as the carrier, potent antitumor antibiotic LDM as the “warhead”, the energized fusion protein VH-LDP-AE was prepared by DNA recombination and molecular reconstitution. The research results of this invention showed that the energized fusion protein VH-LDP-AE, which consisted of VH domain of mAb 3G11 against type IV collagenase and the antitumor antibiotic LDM not only retained the binding activity of intact mAb 3G11 to type IV collagenase, killed tumor cells potently, and inhibited angiogenesis, but also showed antitumor effects in animal experiments. Through searches, we found that VH-LDP-AE, with a molecular mass of 26.2 kDa, is the minimal domain-antibody-based fusion protein with remarkable therapeutic effects in animal experiments ever reported. VH-LDP-AE reaches a new level in molecule down-sizing of antibody-based drugs and is promising in clinical application.

### BRIEF DESCRIPTION OF DRAWINGS

FIG. 1. Restriction enzymatic analysis of recombinant plasmid pET-VH-LDP.

Lane 1, DNA MW markers (DL15,000); lane 2, pET-30a(+); lane 3, recombinant plasmid pET-VH-LDP; 4, pET-30a(+)/NdeI+XhoI; 5, pET-VH-LDP/NdeI+XhoI; 6, pET-VH-LDP/NdeI+BamHI; 7, pET-VH-LDP/BamHI+XhoI; 8, DNA MW markers (DL2,000).

FIG. 2. SDS-PAGE (left) and Western-blot (right) analysis of each fraction of fusion protein 1H-LDP.

Lane 1, low molecular weight marker; lane 2, total proteins of E. coli BL21star™ carrying plasmid pET-30a(+) after IPTG induction; lane 3, total proteins of recombinant E. coli CAMS/HLDFP before IPTG induction; lane 4, total proteins of recombinant E. coli CAMS/HLDFP after IPTG induction; lane 5, medium sample of recombinant E. coli CAMS/HLDFP after IPTG induction; lane 6, periplasmic fraction of recombinant E. coli CAMSIHLDFP after IPTG induction; lane 7, cytoplasmic soluble fraction of recombinant E. coli CAMS/HLDFP after IPTG induction; lane 8, cytoplasmic insoluble fraction of recombinant E. coli CAMS/HLDFP after IPTG induction.

FIG. 3. SDS-PAGE analysis of purification of fusion protein VH-LDP by Immobilized Metal Affinity Chromatography (IMAC).

Lane 1, low molecular weight marker; lane 2, total cell protein; 3, sample before applied to Ni²⁺ column; 4, non-desired proteins not bound to the resin; 5,6, fraction of non-desired proteins washed out by 1× Binding Buffer; 7, fraction of non-desired proteins washed out by 1× washing buffer; 8,9, fusion protein VH-LDP obtained by eluting with 1× Strip Buffer.

FIG. 4. Immunoreactivity of fusion protein VH-LDP with type IV collagenase. ∘, fusion protein VH-LDP; ▴, scFv protein against type IV collagenase.

FIG. 5. Immunoreactivity of fusion protein VH-LDP with human hepatoma SMMC-7721 cells. ∘, fusion protein VH-LDP; ▴, scFv protein against type IV collagenase.

FIG. 6. Immunoreactivity of fusion protein VH-LDP with human oral epidermoid carcinoma KB cells. ∘, fusion protein VH-LDP; ▴, scFv protein directly against type IV collagenase.

FIG. 7. Immunoreactivity of fusion protein VH-LDP with solid tumor of mouse hepatoma 22. A, negative control, with PBS instead of fusion protein VH-LDP as the primary antibody; B Immunol histochemical stain of normal liver section of mice reacted with fusion protein VH-LDP; C, Immunol histochemical stain of hepatoma H 22 section reacted with fusion protein VH-LDP; D, positive control, Immunol histochemical stain of hepatoma H22 section reacted with scFv protein directly against type IV collagenase.

FIG. 8, Gelatin zymography analysis of fusion protein VH-LDP in HT-1080 cells. Lane 1, control, treated with PBS; lane 2, treated with 25 μM of fusion protein VH-LDP; lane 3, treated with 50 μM of fusion protein VH-LDP; lane 4, treated with 100 μM of fusion protein VH-LDP; lane 5, treated with 20 μM of 3G11-scFv.

FIG. 9, Preparation of energized fusion protein VH-LDP-AE. ▴, absorbance at 280 nm; ▪, absorbance at 343 nm.

FIG. 10, Inhibition of bFGF-stimulated angiogenesis by energized fusion protein VH-LDP-AE in CAM assay. A, PBS control, treated with PBS and bFGF; B, positive control, treated with LDM (0.1 μg/egg) and bFGF; C, treated with VH-LDP-AE (0.5 μg/egg) and bFGF.

FIG. 11. Energized fusion protein VH-LDP-AE inhibited the growth of H22 in mice. □, control; ▴, LDM (0.05 mg/kg); Δ, VH-LDP-AE (0.25 mg/kg); x, VH-LDP-AE (0.125 mg/kg); +, mitomycin (1 mg/kg).

FIG. 12: Effects of energized fusion protein VH-LDP-AE plus hydroxylcamptothecin on the proliferation of HT-29 cells. ▴, hydroxylcamptothecin; ●, hydroxylcamptothecin+VH-LDP-AE (1 ng/ml); hydroxylcamptothecin+VH-LDP-AE (3 ng/ml); *CDI<0.9; **CDI<0.8; ***CDI<0.7.

FIG. 13. Effects of energized fusion protein VH-LDP-AE plus 5-fluorouracil on the proliferation of HT-29 cells. ▪, 5-fluorouracil; ●, 5-fluorouracil+VH-LDP-AE (1 ng/ml); *CDI<0.9; **CDI<0.8; ***CDI<0.7.

### EXAMPLE 1

### Cloning of VH Domain Gene and LDM Apoprotein LDP Gene and Construction of Recombinant Plasmid PET-VH-LDP

Recombinant plasmid pKFv1027 and pIJ1027GRGDS contained the VH gene and LDP gene, respectively, and were both constructed by our laboratory (the present laboratory will furnish with the same to the public and provide related document). Vector pGEM-T is from Promega Company, E. coli DH5α is stored at our laboratory. The express vector is from Invitrogen Company and stored at our laboratory. PCR primer is synthesized by Sangon Company with recognition sites for corresponding restriction enzymes (Takara Company) introduced therein.

```
The 5′ primer of VH (PH1, SEQ ID NO: 3):
5′GATA CATATG CAGGTGAAGCTGCAGCAGTCT3′;
                NdeI VH
The 3′ primer of VH (PH2, SEQ ID NO: 4):
5′CATAGGATCCGCCACCGCC TGAGGAGACGGTGACC GTGGT3′
              BamHI spacer VH
The 5′ primer of LDP (PLD1, SEQ ID NO: 5):
5′GATA GGATCC GCGCCCGCCTTCTCCGTCAGT3′
                BamHI LDP
The 3′ primer of LDP3 (PLD2, SEQ ID NO: 6):
5′GTTA CTCGAG GCCGAAGGTCAGAGCCACGTG3′
                XhoI LDP
```

The VH gene fragments with GGGGS flexible spacer sequence added in C-terminal were obtained by PCR reaction wherein the template was recombinant plasmid pKFv1027 and the primer was PH1 and PH2 for 5′ terminal and 3′ terminal, respectively; at the same time, the LDP gene fragments were obtained by PCR amplification in which recombinant plasmid pIJ1027GRGDS was the template and the primer was PLD1 and PLD2 for 5′ terminal and 3′ terminal, respectively.

PCR procedure as described below: 1.94° C. for 2 minutes; 2.25 cycles of: 94° C. for 1 minute, 55° C. for 1 minute, 72° C. for 1 minute; and 3.72° C. for 10 minutes.

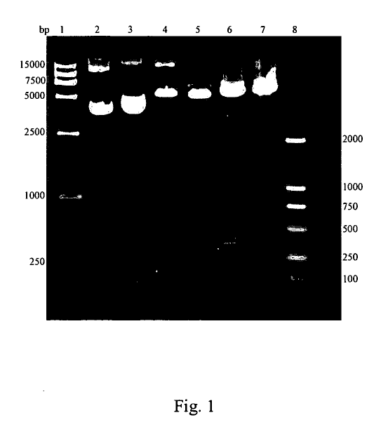

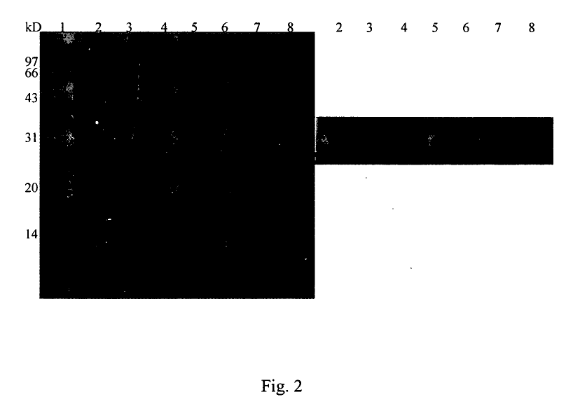

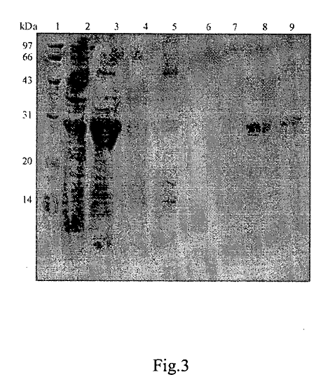

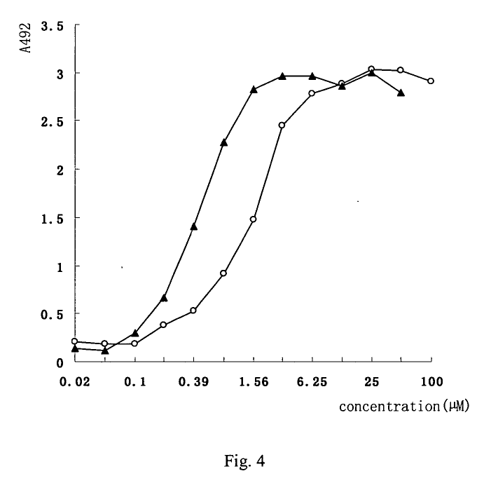

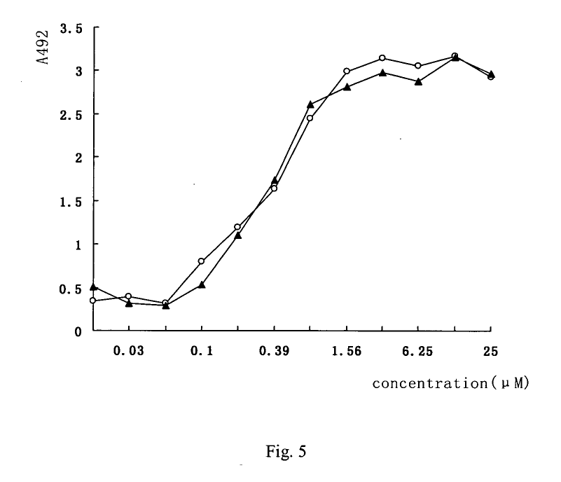

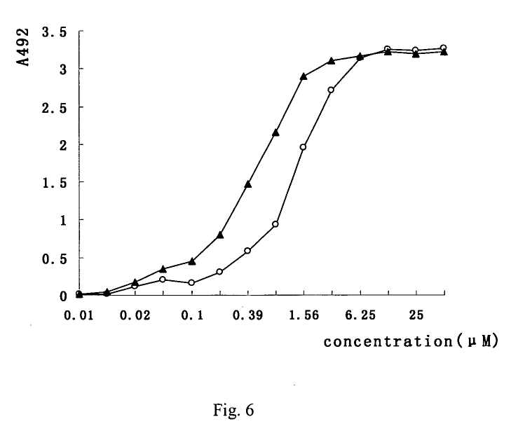

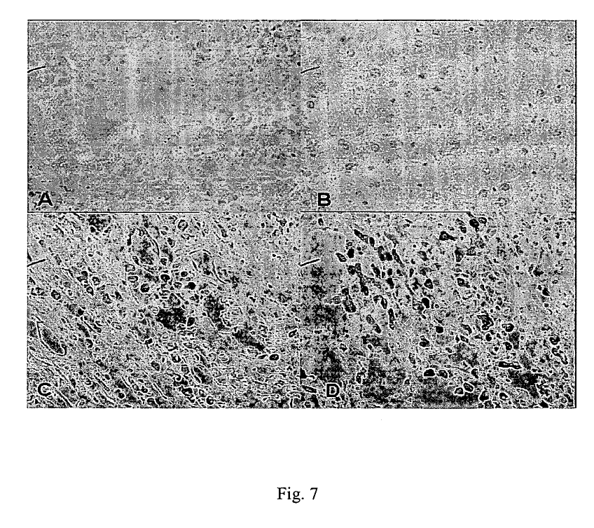

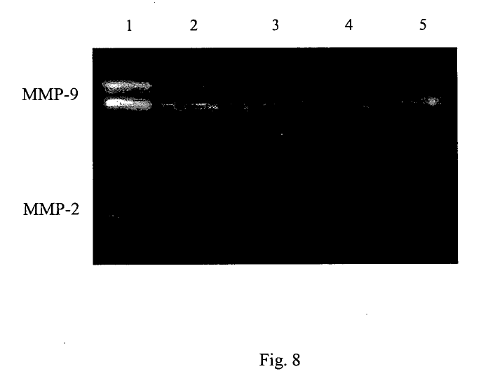

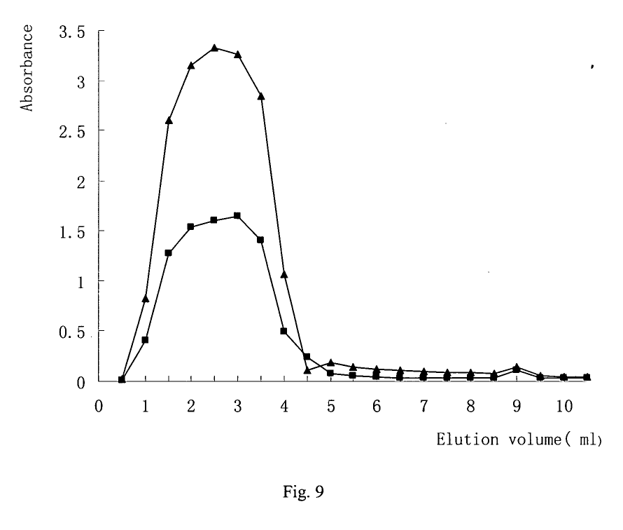

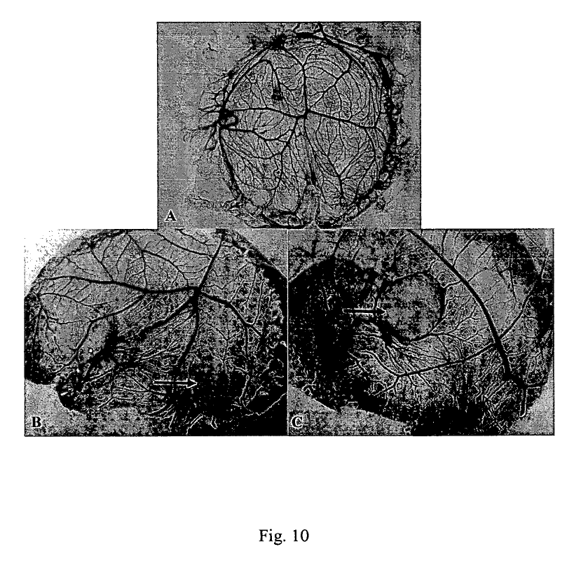

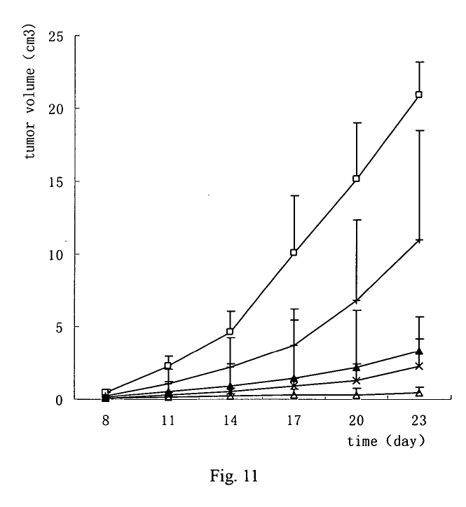

Two PCR products were purified by DNA glass-milk purification kit (BioDev), connected with pGEM-T (Promega), transformed with E. coli DH5α, and then recombinant plasmids are screened. After being confirmed by DNA sequencing (Sangon), the recombinant plasmids were named as pGEM-T-VH and pGEM-T-LDP, respectively. The pGEM-T-VH was digested with BamHI and XhoI, and then the LDP gene fragments were released. The recombinant plasmid pET-LDP were obtained by subcloning LDP gene into the vector pET-30a(+). The pGEM-T-VH was digested with NdeI and BamHI, and the released VH gene fragments were subcloned into vector pET-LDP. And the recombinant expression plasmid pET-VH-LDP were obtained and confirmed by enzyme digestion (FIG. 1) and DNA sequencing. The full length gene of fusion protein was 732 bp and coded for 243 amino acids, wherein VH gene was 360 bp and coded for 120 amino acids, gene of flexible spacer was 15 bp and coded for 5 amino acids, LDP gene was 330 bp and coded for 110 amino acids, XhoI enzyme digestion site is 6 bp and coded for 2 amino acids, his-tag gene was 18 bp and coded for 6 amino acids, and termination codon was 3 bp and coded no amino acid.

In the invention, the XhoI enzyme site was introduced to the 3′-terminal of apoprotein LDP gene of lidamycin, the cluster for 6 successive His residues at multi-cloning site of vector pET-30a(+) would be used well, so there was a His₆-Tag in the 3′-terminal of the fusion protein for its purification and identification.

### EXAMPLE 2

### Inducible Expression of the Fusion Protein VH-LDP in E. Coli BL21star™(DE3)

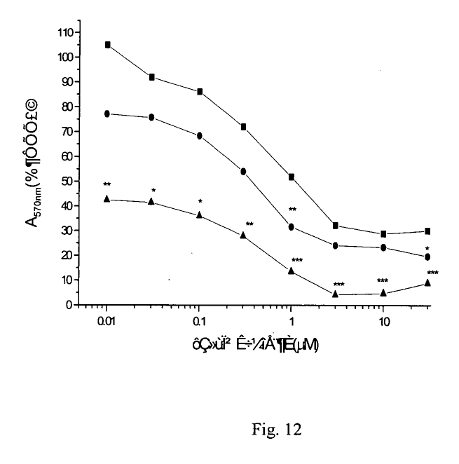

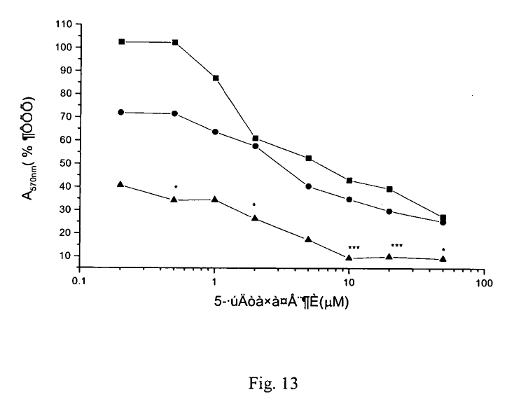

The E. coli strain BL21star™(DE3) in the present invention is from Invitrogen. The recombinant plasmid pET-VH-LDP was transformed into E. coli BL21star™(DE3) and the recombinant transformant was obtained. Single colony of the transformant was transferred into LB medium containing 30 Hg/ml of kanamycin and cultured overnight at 37° C. Next day the strains were inoculated by a volume of 1:50 and cultured at 37° C. until the OD₆₀₀ was 0.7. Isopropyl β-D-thiogalactopyranoside (IPTG) was added to a final concentration of 0.05 mM. After 3 hours of growth, the total cell protein sample, medium sample, periplasmic fraction, cytoplasmic solube fraction, cytoplasmic insoluble fraction (inclusion bodies) of the cultured E. coli were prepared in accordance with pET system manual (Novagen, 9^{th} edition). And the expression of exogenous protein was analyzed by 15% SDS-PAGE under reducing conditions. The results showed that there was exogenous protein expression in the recombinant E. coli after IPTG induction and the expression yield was over 30% of total cell protein. The expression protein was insoluble inclusion bodies of E. coli (FIG. 2).

For Western-blot assay, after SDS-PAGE, the proteins were transblotted onto a PVDF membrane under constant current about 0.65 mA/cm² for 1 hour and 50 minutes. The PVDF membrane was incubated with anti-His₆-Tag mAb diluted (1:2000) in blocking buffer as primary antibody and then with HRP-conjugated goat anti-mouse IgG as secondary antibody, whereupon the membrane was visualized. It was confirmed that the recombinant strain expressed successfully the fusion protein VH-LDP with a His6-Tag in the C-terminal (FIG. 2).

One transformant strain named CAMS/HLDFP that express fusion protein VH-LDP was deposited at China General Microbiological Culture Collection Center (CGMCC, zhongguancun beiyitiao, Beijing) on Apr. 9, 2004 with accession number CGMCC No. 1130.

### EXAMPLE 3

### Purification and Refolding of the Fusion Protein VH-LDP

The fusion protein VH-LDP was purified under denaturing conditions by His-Bind purification kit (Novagen), and the purification process was operated following the user's guide. After being pretreated, the inclusion body sample was loaded to the Ni-NTA column. The column was washed successively with: (1) 10 vol. of binding buffer (5 mM imidazole, 0.5 M NaCl, 20 mM Tris-HCl, 6 M urea, pH 7.9), (2) 6 vol. of washing buffer (60 mM immidazole, 0.5 M NaCl, 20 mM Tris-HCl, 6 M urea, pH 7.9). The purified protein was finally eluted with 6 vol. of eluting buffer (100 mM EDTA, 0.5 M NaCl, 20 mM Tris-HCl, 6 M urea, pH 7.9) (FIG. 3). The theoretical MW of fusion protein VH-LDP was 25.4 kDa.

The fusion protein VH-LDP after purification was refolded: the purified protein was diluted to a final concentration of 15 μM with eluting buffer mentioned above, and β-mercaptoethanol was added to a final concentration of 10 mM. After being stored at room temperature for 30 min, the sample was placed in a dialysis bag and dialyzed against at least 50 vol. of refolding buffer 1 (50 mM Tris-HCl pH 8.0, 1 mM EDTA, 200 mM NaCl, 6 M urea). And the following dialysis was against the same buffer with step-wise reduction in the urea concentration (3M, 2M, 1M, 0.5M, and 0M). On a 1-M stage, 750 μM of glutathione (GSSG) and 400 mM of L-Arginine were added to the dialysis buffer. Then the sample was dialyzed against 50 vol. of PBS solution (pH 7.4). Each dialysis was performed for 12 h and with buffer changed twice. All dialysis was performed at 4° C. After dialysis, the sample was centrifuged at 10,000 g for 30 min at 4° C. and the supernatant was collected. After being condensed, the active fusion protein VH-LDP was obtained and stored at −20° C. for later use.

### EXAMPLE 4

### Immunoreactivity of Fusion Protein VH-LDP with Type IV Collagenase and Tumor Cells

Immunoreactivity of fusion protein VH-LDP was detected by ELISA. At first, type IV collagenase or tumor cells was coated or fixed to 96-well ELISA plates: well was coated with 100 μl of type IV collagenase (10 μg/ml, diluted by PBS) and the plates were stored at 4° C. overnight; Human hepatoma SMMC-7721 cells or human KB cells were seeded at 10⁴/well in 96-well plates and cultured for 24 h, washed with PBS for 3 times, added 0.05% cold (4° C.) Glutaraldehyde and fixed for 15 min. Then the coated or fixed plates were washed with PBS 3 times, and blocked with 200 μl/well of 1% bovine serum albumin (BSA)/PBS at 4° C. overnight. The plates were washed 3 times with PBS. Then 200 μl of serial concentrations of fusion protein VH-LDP were added into each well of the plates and incubated for 2 hours at 37° C. Washing the plates 3 times with 0.05% Tween 20/PBS (PBST), adding 50 μl of anti-His₆-Tag monoclonal antibody diluted 1:1500 to each well, and incubating at 37° C. for 1 h. Washing the plates 3 times with PBST, adding 50 μl of HRP conjugated goat anti-mouse IgG (at 1:2000 dilution), and incubating at 37° C. for 1 hour. Washing the plates with PBST 6 times, adding 100 μl of OPD substrate reaction solution to each well, incubating the plates at room temperature for 10 min in the dark. Stopping the reaction with 100 μl of H₂SO₄ (2 mol/L). Then the optical density (OD) of each well was measured at 490 nm using a microplate reader.

The results demonstrated that the binding activities of fusion protein VH-LDP to type IV collagenase (FIG. 4), human hepatoma SMMC-7721 cells (FIG. 5), and human oral epidermoid carcinoma KB cells (FIG. 6) were all positive.

### EXAMPLE 5

### Immunoreactivity of the Fusion Protein VH-LDP with the Solid Tumor of Mouse Hepatoma 22

Immunohistochemical (IHC) staining was performed by streptavidin-biotin-peroxidase complex (SABC) staining with SABC kit (Boster Company). The sections were blocked by normal goat serum for 20 min at room temperature and the excessive liquid was removed. The sections without washing were added with diluted said VH-LDP fusion protein as prepared in example 3 and incubated at room temperature; then diluted anti-His₆-tag antibody and biotinylated goat-anti-mouse IgG were added in turn. Finally SABC reagent was added. DAB kit was used as a chromogen for visualized reaction at room temperature. The sections were counter-stained by hematoxylin, dehydrated, cleared, sealed, and observed with microscope. The results showed that fusion protein VH-LDP was positively stained in mouse hepatoma sections, and negatively stained in mouse normal liver sections (FIG. 7). This indicated that VH-LDP has selectivity to tumor tissues.

### EXAMPLE 6

### Inhibition of Type IV Collagenase Activity by Fusion Protein VH-LDP

Gelatin zymography protocol was used. Exponentially growing HT-1080 cells were seeded at 10⁵/well in 24-well plates and incubated at 37° C. in 5% CO₂. After 24 h, the culture medium was removed and 1 ml serum-free RPMI 1640 medium was added for washing twice. Add 120 μl of serum-free RPMI 1640 medium and 30 μl of fusion protein VH-LDP as prepared in example 3 to each well, and add 30 μl PBS to control well. Culturing the cells at 37° C. for 24 h. Harvest the culture medium, and centrifuging at 500 g for 5 min. Then take the supernatant for non-denaturalization electrophoresis on SDS-PAGE. Wash the gels with distilled water 3 times. Put the gels into 100 ml of 2.5% TritonX-100, shake on a shaker for 30 min at low speed. Then Wash the gels 2 times with distilled water; repeat the washing process with TritonX-100 above. The gels was washed with distilled water 2 times, and incubated at 37° C. for 16-18 h in 100 ml of gelatinase buffer (50 mM Tris-HCl, pH 7.5, 200 mM NaCl, 10 mM CaCl₂, 1 μM ZnCl₂). The gels were stained with Coomassie-brilliant blue R-250 and destained with acetic acid: methanol:water (10:45:45). The negative stained bands were observed.

The results showed that fusion protein VH-LDP could inhibit significantly the activity of type IV collagenase secreted by cancer cells. Compared with the control, the negative stained bands for type IV collagenase was weakened, and the extent of inhibition was dependent on the drug concentration (FIG. 8).

### EXAMPLE 7

### Preparation of Lidamycin (LDM)

The LDM-producing strain (with accession number CGMCC NO. 0135, published in Chinese Patent application 00121527.2) in the frozen-dried tube was resuspended in 0.7 ml salt-free water, transplanted into the Gause's NO. 1 slant medium and grown at 28° C. for about 7-10 days. Then the aerial mycelium of the strain was transferred to 100/500 ml flask containing the following medium: 1% starch, 0.5% corn syrup, 0.5% peptone, 0.5% glucose, 0.02% MgSO₄, 0.06% KI, 1.5% corn starch, 0.4% CaCO₃, pH 7.0. Shaking fermentation for 48 h at 28° C. then transferred to 1000/5000 ml flask by the volume of 5% and shacked about 18 h at 28° C. in the same medium. After that the producer was transferred to a 200 L fermentation tank containing 100 L of medium by the volume of 2% and 0.03% defoaming agent was added. The fermentation parameters include: pressure, 0.04; temperature, 28° C.; stirring speed, 400 rpm, air flow 1/1, pH 6.5-7.0, fermentation time, 96 hours. 10 L fermented liquid was centrifuged and the pH of the supernatant was adjusted to 4.0 using HCl. Then 4.5 kg of (NH₄)₂SO₄ was added, and the liquid was stirred for 3 h at 8° C. The precipitated LDM was separated by centrifugation at 8000 rpm at 4° C. for 15 min. The pellet was dissolved in 200 ml cold water and dialyzed. Then the unsolvable sediment was removed by centrifugation. The supernatant was absorbed by hydroxyl apatite column, eluted by 0.001M PBS (pH6.8) and frozen-dried. Then 1500 mg crude product dissolved in water was loaded to Sephadex G-75 column. The active part was frozen-dried. The refined LDM product with high anti-tumor activity was about 145 mg.

### EXAMPLE 8

### Preparation of Energized Fusion Protein VH-LDP-AE

10 mg of frozen-dried LDM with high activity as obtained in Example 7 were added into 5 ml cold methanol and shaken for 5 min, placed at −20° C. for 1 hour. Shake once during the course. Centrifuge at 12,000 r/min at 0° C. for 20 min. The supernatant was rich in chromphore and the sediment contained the apoprotein. The extraction was repeated twice. Chromophore in methanol was vapored and concentrated, stored at −70° C. The chromophores were labile, and the experiments were performed at 4° C. and prevented from illumination. Fusion protein VH-LDP from example 3 was dissolved in PBS (0.01 M, pH 7.4), and 5 times of chromophore-in-methanol by molecular ratio were added to the VH-LDP-containing PBS solution with the volume ratio of 1:50. After shaking, the mixture was placed at room temperature for 12 hours. Then separation and purification were performed using PD-10 (Sephadex G-25 column, Pharmacia) and detected the absorbance at 280 nm and 343 nm. Then the energized fusion protein VH-LDP-AE was collected (FIG. 9). The theoretical molecular weight of VH-LDP-AE was 26.2 kDa.

### EXAMPLE 9

### Cytotoxicity of Energized Fusion Protein on Cancer Cells In Vitro

MTT assay was used. After digestion and counting, cells in exponential growth phase were seeded at 3000 cells/well in 96-well plates, and cultured at 37° C. in 5% CO₂ for 24 hours. Drugs of various concentrations were added with 3 aliquots, and the cells were cultured for another 72 h. 50 μl of MTT (2 mg/ml) in serum-free RPMI 1640 were added into each well, and the cells were cultured at 37° C. for 4 h. Remove the culture medium gently; add 150 μl DMSO each well; incubate at room temperature for 15 min on a shaker; measure the absorbance at 560 nm on a microplate reader. Survival ratio and IC₅₀ values were calculated according to the following equation: Survival ratio=(Aₜₑₛₜ−A_{blank})/(A_{control}−A_{blank})×100%. The results indicated that energized fusion protein VH-LDP-AE displayed extremely potent cytotoxicity to cancer cells. As table 1 demonstrated, the IC₅₀ values to HT-1080 cells, KB cells, and PG cells were all lower than 1×10¹¹ M (Table 1).

TABLE 1
The cytotoxicity of VH-LDP-AE to cancer cells

| | IC₅₀(M) | | |
|---|---|---|---|
| Groups | HT-1080 | KB | PG |
| LDM | 2.21 × 10⁻¹² | 1.08 × 10⁻¹² | 6.30 × 10⁻¹² |
| VH-LDP-AE | 7.65 × 10⁻¹³ | 4.35 × 10⁻¹⁴ | 1.49 × 10⁻¹³ |

### EXAMPLE 10

### Anti-Angiogenic Activity of the Energized Fusion Protein VH-LDP-AE

Anti-angiogenic activity of VH-LDP-AE was examined in chick embryoallantoic membrane assay (CAM). The surface of 7-day-old post-fertilization chick eggs (White Leghorn) in a 60% humidified incubator at 37° C. was sterilized and the CAM was 2) exposed by cutting a window (2 cm²) on the egg shell using the false air-sac technique. After 24 h, 10 μl of bFGF was dipply added to agarose disks with the energized fusion protein VH-LDP-AE at various concentrations which was prepared as mentioned in Example 7, and then the disks were placed on top of the CAM. After the windows were sealed with transparent tape, the eggs were incubated for further 72 h. The results shown in FIG. 10 indicated that VH-LDP-AE significantly suppress angiogenesis stimulated by bFGF.

### EXAMPLE 11

### The Therapeutic Effects of Energized Fusion Protein VH-LDP-AE on the Growth of Transplanted Hepatoma 22 in Mice

Kunming mice, weighing between 18-22 g, were randomly separated into different groups of 10 mice each. On day 0, hepatoma 22 cells (1.5×10⁶ cells/0.2 ml/mouse) diluted with saline were transplanted subcutaneously into the right axilla of mice. On day 1 (after 24 h), the therapy was started and VH-LDP-AE at different doses, LDM at 0.05 mg/kg, mitomycin (MMC) at 1 mg/kg were administered respectively to the tumor bearing mice (0.2 ml/mouse) by injection into the tail vein. Mice of control group were injected with saline. Diameters of the tumors were measured every 3 days during the experiment. Weights of the mice were recorded. The tumor volumes were calculated with the following formula: V=0.5ab², where a and b is the long and the perpendicular short diameters of the tumor, respectively. The curves of tumor growth were plotted and the inhibition rates were calculated.

The results showed that both 0.25 mg/kg and 0.125 mg/kg of energized fusion protein VH-LDP-AE inhibited or retarded the growth of hepatoma 22 in mice, and the inhibition efficacy was more potent than the free lidamycin at the maximal tolerated dose of 0.05 mg/kg. This indicated that VH-LDP-AE can enhance the therapeutic effects of lidamycin (FIG. 11). The results on day 14 were shown on Table 2:

TABLE 2
Inhibition of the growth of transplantable hepatoma 22 by VH-LDP-AE in mice

| | Doses | Mice number | Body weight (g) | Tumor volume (cm³) | Inhibition |
|---|---|---|---|---|---|
| | (mg/kg) | begin/end | begin/end | x̅ ± s | rate (%) |
| Control | | 10/10 | 18.89/33.20 | 4.60 ± 1.48 | |
| LDM | 0.05 | 10/10 | 19.77/26.28 | 0.94 ± 0.53 | 79.6^{ΔΔ} |
| VH-LDP-AE | 0.25 | 10/10 | 19.27/23.50 | 0.19 ± 0.21 | 95.9**^{ΔΔ} |
| | 0.125 | 10/10 | 19.01/27.59 | 0.55 ± 0.24 | 88.1*^{ΔΔ} |
| MMC | 1 | 10/10 | 18.82/29.29 | 2.23 ± 2.15 | 51.5^{Δ} |

P < 0.05 vs. LDM, indicated by *;
P < 0.01 vs. LDM, indicated by **
P < 0.05 vs. control, indicated by Δ;
P < 0.01 vs. control, indicated by ΔΔ

The inhibition rates of tumor growth of VH-LDP-AE was 95.9% and 88.1%, at doses of 0.25 mg/kg and 0.125 mg/kg, respectively, the effects were more potent than that of LDM (79.6%), and mitomycin, used as chemotherapeutic drug in clinics, showed the inhibition rate of 51.5% (Table 2). No body weight loss and other severe side-effects were found during the experiments. This indicates that mice well tolerated the doses.

### EXAMPLE 12

### The Inhibitory Effects of VH-LDP-AE in Combination with Various Anticancer Drugs on Cancer Cell Proliferation

MTT assay was used. After trypsin-EDTA digestion, the human colon carcinoma HT-29 cells in exponential growth were seeded at 4000 cells/well in 96-well plates, and cultured for 24 hours. Then add 20 μl of different concentrations of hydroxylcamptothecin or 5-fluorouracil; 8 hours later, add different concentrations of VH-LDP-AE. Continue to culture the cells at 37° C. in 5% CO₂ for 72 h. Add 50 μl of MTT at concentration of 2 mg/ml, culture the cells at 37° C. for 4 hours. Then remove the supernatant; add 150 μl DMSO each well; ten minutes later, measure the absorbance at 560 nm on a microplate reader. In cancer pharmacology, interactions of drug combination were evaluated by coefficient of drug interaction (CDI). There existed synergistic effects with CDI<1, and there existed very significant ones with CDI<0.7. For the combination of hydroxylcamptothecin (1 μM) and energized fusion protein VH-LDP-AE (3 ng/ml), CDI value was less than 0.7, and which indicated that they can inhibited synergistically the proliferation of HT-29 cells (FIG. 12). For 5-fluorouracil plus VH-LDP-AE, the CDI of 10 μM of 5-fluorouracil and 3 ng/ml of VH-LDP-AE was less than 0.7, and which showed that there existed a very obvious synergistic effects between them (FIG. 13). It was usual that monoclonal antibodies were combined with anti-cancer drugs. The present results showed that there existed significantly synergistic effects between energized fusion protein VH-LDP-AE and 5-fluorouracil or hydroxylcamptothecin.

1. A single domain antibody energized fusion protein VH-LDP-AE consisting of a fusion protein VH-LDP that contains the heavy chain variable domain VH of monoclonal antibody 3G11 against type IV collagenase, the flexible spacer GGGGS, the apoprotein of lidamycin (LDP), and a His₆-tag tail; and an active enediyne chromophore (AE) that derives from lidamycin.

2. The single domain antibody energized fusion protein VH-LDP-AE of claim 1, wherein the gene sequence coding for said fusion protein VH-LDP is set forth in SEQ ID NO: 1, the amino acid sequence thereof is set forth in SEQ ID No: 2.

3. The single domain antibody energized fusion protein VH-LDP-AE of claim 1, wherein the molecular ratio of said fusion protein and active chromophore AE of lidamycin is 1:1.

4. A method for producing single domain antibody energized protein VH-LDP-AE of claim 1, comprising:
1) Preparing the fusion protein VH-LDP containing single domain antibody against type IV collagenase;
2) Preparing the active chromophore AE of lidamycin by the method of cold methanol extraction;
3) Mixing the resultant active chromophore AE of lidamycin extracted by cold methanol in solution of methanol and VH-LDP in 0.01 M PBS (pH7.4) by a molecular ratio of 1:5 and a volume ratio of 50:1, reacting in dark at room temperature for 12 hours, and the energized fusion protein VH-LDP-AE is obtained.

5. Use of single domain antibody energized fusion protein VH-LDP-AE of claim 1 in manufacture of anti-angiogenic and anti-cancer targeting drug.

6. The use of claim 5, wherein said tumor is selected from the group consisting of solid tumors including digestive tract cancers, such as hepato-carcinoma, colon carcinoma, rectum carcinoma, esophageal carcinoma, gastric carcinoma; breast carcinoma; ovarian carcinoma; lung carcinoma; and renal carcinoma.

7. A pharmaceutical composition comprising therapeutically effective amount of single domain antibody energized fusion protein VH-LDP-AE of claim 1, and optionally, pharmaceutical acceptable carrier and/or excipient.

8. A method for treating a tumors in a human comprising the step of administering a therapeutically effective amount of single domain antibody energized fusion protein of claim 1 to a patient with the tumor.

9. A method for treating a tumors in a human comprising the step of administering a therapeutically effective amount of said pharmaceutical composition of claim 7 to a patient with the tumor.
